(19) United States
(12) Patent Application Publication
(10) Pub. No.: US 2009/0221058 A1
(43) Pub. Date: Sep. 3, 2009

(54) Potentiometric biosensor for detection of lactate in food and forming method thereof

(75) Inventors: Shen-Kan Hsiung, Tao-Yuan (TW); Jung-Chuan Chou, Tao-Yuan (TW); Tai-Ping Sun, Tao-Yuan (TW); Nien-Hsuan Chou, Tao-Yuan (TW); Zhi-Cheng Lin, Tao-Yuan (TW)

Correspondence Address: WPAT, PC, 7225 BEVERLY ST., ANNANDALE, VA, 22003, US

(73) Assignee: CHUNG YUAN CHRISTIAN UNIVERSITY, Tao-Yuan (TW)

(21) Appl. No.: 12/040,237

(22) Filed: Feb. 29, 2008

(51) Int. Cl. C12M 1/40; C23C 14/08; C23C 14/35; B05D 5/00

(52) U.S. Cl. 435/287.1; 427/58; 204/192.15

(57) ABSTRACT

The present invention discloses a potentiometric biosensor for detecting lactate in food, and the forming method thereof. The disclosed biosensor comprises a substrate, and conducting layer on the substrate, an oxide layer on the conducting layer, and an enzyme layer on the oxide layer, wherein the enzyme layer comprises Lactate dehydrogenase (LDH). The detection signal is transmitted for further processing through a wire connected to the conducting layer, or a window formed on the surface of conducting layer.

### BACKGROUND OF THE INVENTION

1. Field of the Invention

The present invention is generally related to biosensors and the fabrication method thereof, and more particularly, a potentiometric biosensor for detection of lactate in food and the forming method thereof.

2. Description of the Prior Art

Biosensor is commonly defined as an analytical device which combines energy converter with immobilized biomolecules for detecting specific chemicals via the interaction between biomolecules and such specific chemicals. The above-mentioned energy converter can be a potentiometer, a galvanometer, an optical fiber, a surface plasma resonance, a field-effect transistor, a piezoelectric quartz crystal, a surface acoustic wave, and so on. The field-effect transistor which can be fabricated to form the miniaturized component via mature semiconductor process has become an important technique for developing light and portable products, which is the current market trend.

At present, the commercial biosensors based on field-effect transistors detect specific chemicals utilizing amperometeric technology. The principle of amperometeric technology is detecting a small electric current in organisms. Amperometric biosensors have fast response, but the read circuit needs an additional bias voltage to convert the signals. Therefore, the fabrication of amperometric biosensors requires a more complicated design and higher costs. A redox reaction occurs when the amperometric biosensors detect specific chemicals via the interaction between biomolecules and such specific chemicals, and it produces a small electric current which flows through the surface of sensor window, which would destroy biological molecules (such as enzymes), and hence affect the follow-up use of enzymes regarding chemical response capability. Moreover, the biosensors based on field-effect transistors are mostly produced by the semiconductor manufacturing process that needs strict conditions (such as the need for high vacuum environment, etc.), which results in high costs of production.

On other hand, most of the commercial biosensors are developed for medical purpose (such as measurement of the lactate concentration in human blood), but the biosensors for food-related testing which are significant to human health is absent. How to make the biosensors having simple structure, good stability, and replaceable with low cost has become the current trend in sensor development.

### SUMMARY OF THE INVENTION

In accordance with the present invention, a potentiometric biosensor for detection of lactate in food and forming method thereof is provided.

The present invention further discloses a potentiometric biosensor for detection of lactate in food. The potentiometric biosensor revealed in this invention is for detecting the content of lactic in the food and judging the freshness of the food.

The present invention discloses a potentiometric biosensor based on field-effect transistors which can be fabricated to form the miniaturized component via semiconductor process. A potentiometric biosensor doesn't need an additional bias voltage to convert the signals. The disclosed biosensor comprises a substrate, and conducting layer on the substrate, an oxide layer on the conducting layer, and an enzyme layer on the oxide layer, wherein the enzyme layer comprises Lactate dehydrogenase (LDH). The detection signal is transmitted for further processing through a wire connected to the conducting layer, or a window formed on the surface of conducting layer. The disclosed biosensor is replaceable.

### BRIEF DESCRIPTION OF THE DRAWINGS

FIG. 1 is a schematic diagram of the potentiometric biosensor for detection of lactate in food according to the first embodiment of the present invention;

FIG. 2 is a schematic diagram of the potentiometric biosensor for detection of lactate in food according to the second embodiment of the present invention;

FIG. 3 is a schematic diagram of the potentiometric biosensor for detection of lactate in food according to the third embodiment of the present invention;

FIG. 4 is a schematic diagram of the potentiometric biosensor for detection of lactate in food according to the fourth embodiment of the present invention;

FIG. 5 is a flow chart of the method for forming a potentiometric biosensor to detect lactate in food according to the present invention;

FIG. 6A is a schematic diagram of the potentiometric biosensor for detection of lactate in food according to an example of the fifth embodiment of the present invention;

FIG. 6B is a schematic diagram of the potentiometric biosensor for detection of lactate in food according to another example of the fifth embodiment of the present invention;

### DESCRIPTION OF THE PREFERRED EMBODIMENTS

What is probed into the invention is a potentiometric biosensor for detection of lactate in food. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following specification. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

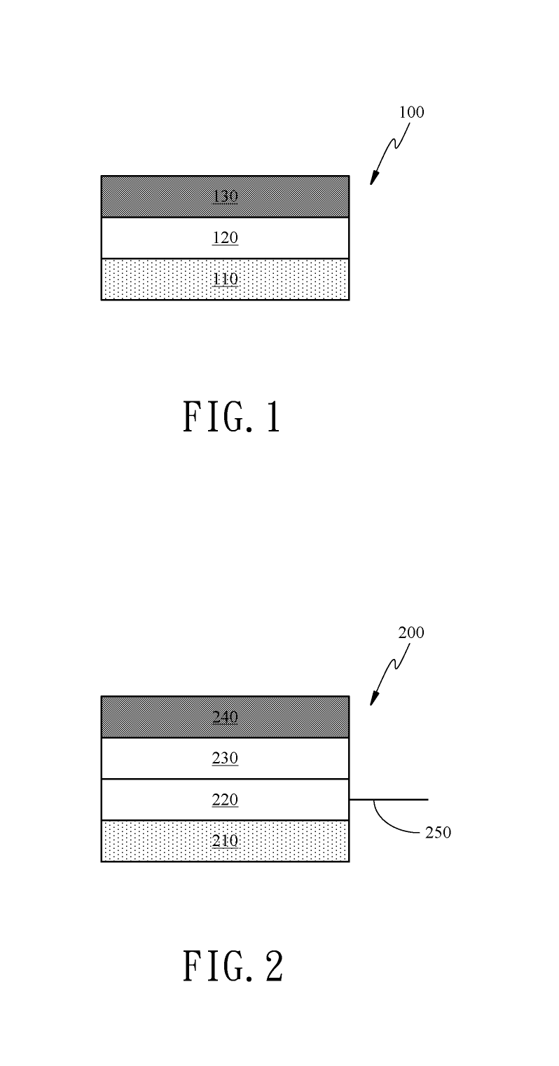

As shown in FIG. 1, a first embodiment of the present invention discloses a potentiometric biosensor 100 for detection of lactate in food, comprising a substrate 110, an oxide layer 120 on the substrate 110, and an enzyme layer 130 on the oxide layer 120. The material of above-mentioned substrate 110 comprises one selected from the group consisting of the following: insulating materials (such as insulating glass), non-insulated materials (such as indium-tin oxide glass and non-insulated tin oxide glass) and polyethylene terephthalate (PET). The above-mentioned oxide layer 120 is non-insulated solid ion, such as tin oxide and so on. The above-mentioned enzyme layer 130 comprises Lactate dehydrogenase (LDH).

As shown in FIG. 2, a second embodiment of the present invention discloses a potentiometric biosensor 200 for detection of lactate in food, comprising a substrate 210, a conducting layer 220 on the substrate 210, an oxide layer 230 on the conducting layer 220, and an enzyme layer 240 on the oxide layer 230. An example of this embodiment is shown that the potentiometric biosensor 200 further comprises a wire 250 connected to the conducting layer 220 to facilitate the transmission of the detection signal. The above-mentioned conducting layer 220 possesses a low impedance to enhance the transmission efficiency of the detection signal, and the conducting layer 220 comprises one selected from the group consisting of the following: copper, carbon, silver, aurum, silver chloride, Indium tin oxides (ITO). The above-mentioned wire 250 comprises one selected from the group consisting of the following: copper, carbon, silver, aurum, silver chloride, Indium tin oxides (ITO). The material of above-mentioned substrate 110 comprises one selected from the group consisting of the following: insulating materials (such as insulating glass), non-insulated materials (such as indium-tin oxide glass and non-insulated tin oxide glass) and polyethylene terephthalate (PET). The above-mentioned oxide layer 230 is non-insulated solid ion, such as tin oxide and so on. The above-mentioned enzyme layer 240 comprises Lactate dehydrogenase (LDH).

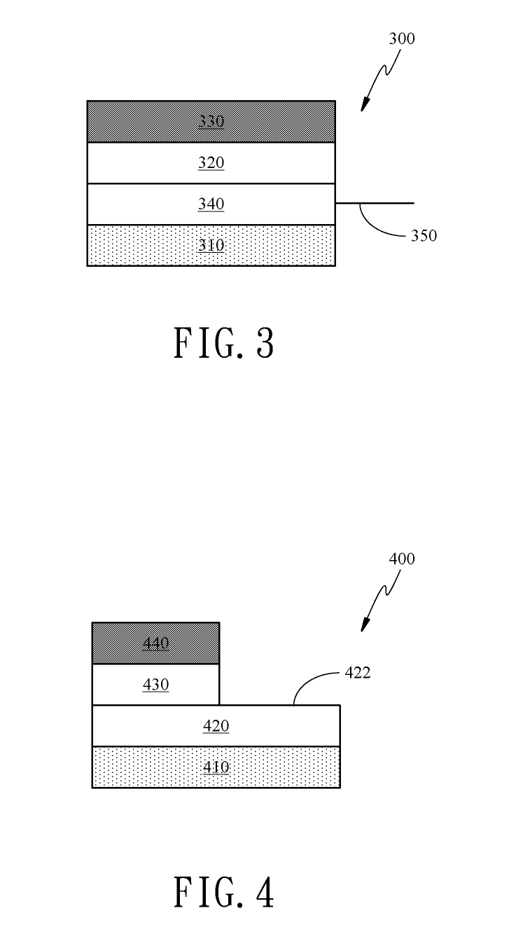

As shown in FIG. 3, a third embodiment of the present invention discloses a potentiometric biosensor 300 for detection of lactate in food, comprising a substrate 310, an oxide layer 320 on the substrate 310, and an enzyme layer 330 on the oxide layer 320. The above-mentioned enzyme layer 330 is immobilized on the oxide layer 320 via covalent bonding by 3-glycidoxypropyltrimethoxysilane (GPTS). The material of above-mentioned substrate 110 comprises one selected from the group consisting of the following: insulating materials (such as insulating glass), non-insulated materials (such as indium-tin oxide glass and non-insulated tin oxide glass) and polyethylene terephthalate (PET). The above-mentioned oxide layer 320 is non-insulated solid ion, such as tin oxide and so on. The above-mentioned enzyme layer 330 comprises Lactate dehydrogenase (LDH).

An example of this embodiment is shown that the potentiometric biosensor 300 further comprises a conducting layer 340 which lies between the substrate 310 and the oxide layer 320 for outward transmission of detection signal. In addition, the potentiometric biosensor 300 further comprises a wire 350 connected to the conducting layer 340 to facilitate the transmission of the detection signal. The conducting layer 340 comprises one selected from the group consisting of the following: copper, carbon, silver, aurum, silver chloride, Indium tin oxides (ITO). The above-mentioned wire 350 comprises one selected from the group consisting of the following: copper, carbon, silver, aurum, silver chloride, Indium tin oxides (ITO).

As shown in FIG. 4, a fourth embodiment of the present invention discloses a potentiometric biosensor 400 for detection of lactate in food, comprising a substrate 410, a conducting layer 420 on the substrate 410, an oxide layer 430 on the conducting layer 420, and an enzyme layer 430 on the oxide layer 420. The above-mentioned conducting layer 420 comprises an exposed surface to electrically couple with the external world and for outward transmission of detection signal. The material of above-mentioned substrate 110 comprises one selected from the group consisting of the following: insulating materials (such as insulating glass), non-insulated materials (such as indium-tin oxide glass and non-insulated tin oxide glass) and polyethylene terephthalate (PET). The conducting layer 420 comprises one selected from the group consisting of the following: copper, carbon, silver, aurum, silver chloride, Indium tin oxides (ITO). The above-mentioned oxide layer 430 is non-insulated solid ion, such as tin oxide and so on. The above-mentioned enzyme layer 440 comprises Lactate dehydrogenase (LDH).

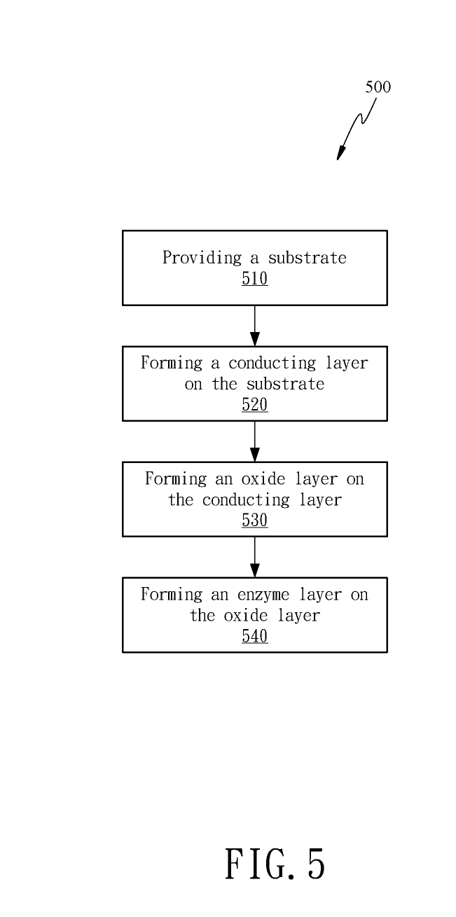

As shown in FIG. 5, the present invention discloses a method, flow chart 500, for forming a potentiometric biosensor to detect lactate in food. The flow chart 500 comprises for four major steps. The first step 510 is providing a substrate, and the second step 520 is forming a conducting layer on the substrate, and the third step 530 is forming an oxide layer on the conducting layer, and the fourth step 540 is forming an enzyme layer on the oxide layer. An example of this embodiment is shown that the method for forming a potentiometric biosensor further comprises providing a wire after the formation of the conducting layer on the substrate, the wire being connected to the conducting layer for the transmission of detection signal. Moreover, another example of this embodiment is shown that the method for forming a potentiometric biosensor further comprises the step of, after the formation of the conducting layer on the substrate, forming an exposed surface on the conducting layer for the transmission of the detection signal. The above-mentioned enzyme layer is immobilized by covalent bonding method or entrapment method. The enzyme layer is immobilized on the oxide layer via covalent bonding by 3-glycidoxypropyltrimethoxysilane (GPTS) at 150 degrees Celsius for about 2 hours. The enzyme layer comprises deionized water, dipotassium hydrogen phosphate, lactic dehydrogenase. The oxide layer is formed by deposition of tin oxide on the substrate through magnetron sputtering at radio frequency (RF) power 50 W for 40 minutes.

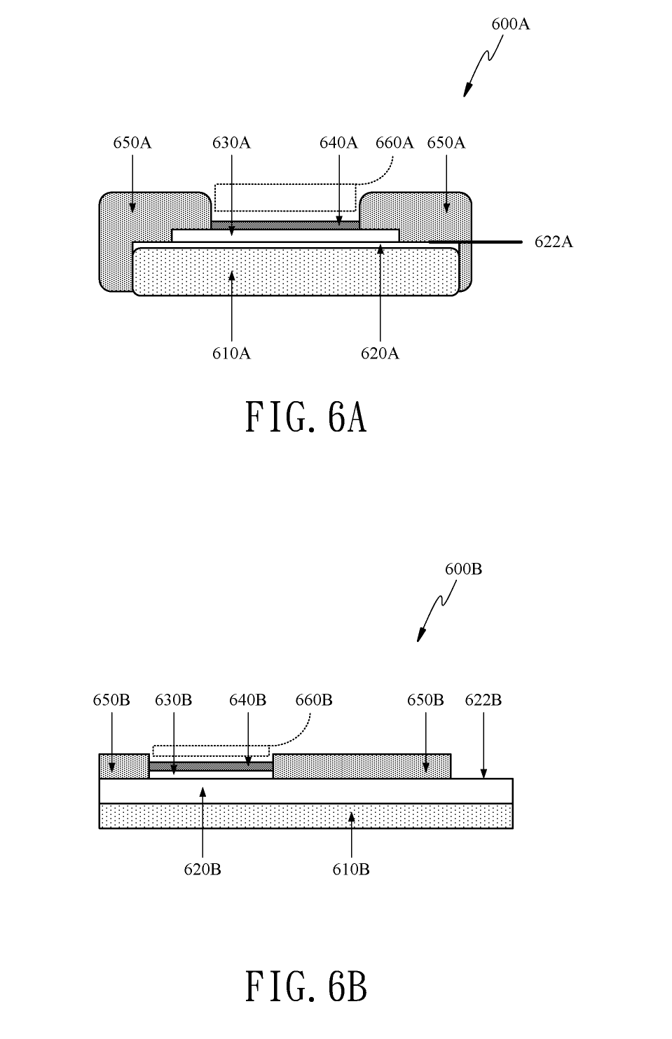

As shown in FIG. 6, a fifth embodiment of the invention discloses a potentiometric biosensor (600A; 600B) for detection of lactate in food, comprising a substrate (610A; 610B), a conducting layer (620A; 620B) on the substrate, an oxide layer (630A; 630B) on the conducting layer, an enzyme layer (640A; 640B) on the oxide layer, sealing layer (650A; 650B) on the enzyme layer. The sealing layer (650A; 650B) is to enclose the formed biosensor wherein the sealing layer has a window (660A; 660B) for detection of lactate. The enzyme layer is immobilized on the oxide layer via covalent bonding by 3-glycidoxypropyltrimethoxysilane (GPTS). The sealing layer (650A; 650B) is epoxy resin.

As shown in FIG. 6A, according to an example of this embodiment is shown that the biosensor further comprises a wire 622A connected to the conducting layer to facilitate the transmission of the detection signal. On other hand, as shown in FIG. 6B, according to another example of this embodiment is shown that the conducting layer comprises an exposed surface 622B to electrically couple with the external world and for outward transmission of the detection signal.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A potentiometric biosensor for detecting lactate in food, comprising:
a substrate;
an oxide layer formed on said substrate; and
an enzyme layer formed on said oxide layer.

2. The potentiometric biosensor for detecting lactate in food according to claim 1, wherein said substrate comprises one selected from the group consisting of the following: insulating glass, non-insulated indium-tin oxide glass, non-insulated tin oxide glass and polyethylene terephthalate (PET).

3. The potentiometric biosensor for detecting lactate in food according to claim 1, wherein said oxide layer is tin dioxide.

4. The potentiometric biosensor for detecting lactate in food according to claim 1, wherein said enzyme layer comprises Lactate dehydrogenase (LDH).

5. The potentiometric biosensor for detecting lactate in food according to claim 1, wherein said biosensor further comprises a conducting layer which lies between said substrate and said oxide layer for outward transmission of detection signal.

6. The potentiometric biosensor for detecting lactate in food according to claim 5, wherein said biosensor further comprises a wire connected to said conducting layer to facilitate the transmission of said detection signal.

7. The potentiometric biosensor for detecting lactate in food according to claim 5, wherein said conducting layer possesses a low impedance to enhance detection signal transmission efficiency, and said conducting layer comprises one selected from the group consisting of the following: copper, carbon, silver, aurum, silver chloride, Indium tin oxides (ITO).

8. The potentiometric biosensor for detecting lactate in food according to claim 6, wherein said wire comprises one selected from the group consisting of the following: copper, carbon, silver, aurum, silver chloride, Indium tin oxides (ITO).

9. The potentiometric biosensor for detecting lactate in food according to claim 1, wherein said enzyme layer is immobilized on said oxide layer via covalent bonding by 3-glycidoxypropyltrimethoxysilane (GPTS).

10. The potentiometric biosensor for detecting lactate in food according to claim 5, wherein said conducting layer comprises an exposed surface to electrically couple with the external world and for outward transmission of said detection signal.

11. The potentiometric biosensor for detecting lactate in food according to claim 5, wherein said biosensor further comprises a sealing layer to enclose the formed biosensor wherein said sealing layer has a window for detection of lactate.

12. The potentiometric biosensor for detecting lactate in food according to claim 11, wherein said sealing layer is epoxy resin.

13. A method for forming a potentiometric biosensor to detect lactate in food, comprising:
providing a substrate;
forming a conducting layer on said substrate;
forming an oxide layer on said conducting layer; and
forming an enzyme layer on said oxide layer.

14. The method for forming a potentiometric biosensor to detect lactate in food according to claim 13, further comprising providing a wire after the formation of said conducting layer on said substrate, said wire being connected to said conducting layer for the transmission of detection signal.

15. The method for forming a potentiometric biosensor to detect lactate in food according to claim 13, further comprising after the formation of said conducting layer on said substrate, forming an exposed surface on said conducting layer for the transmission of detection signal.

16. The method for forming a potentiometric biosensor to detect lactate in food according to claim 13, wherein said enzyme layer is immobilized by entrapment method.

18. The method for forming a potentiometric biosensor to detect lactate in food according to claim 13, wherein said enzyme layer is immobilized on said oxide layer via covalent bonding by 3-glycidoxypropyltrimethoxysilane (GPTS) at 150 degrees Celsius for about 2 hours.

19. The method for forming a potentiometric biosensor to detect lactate in food according to claim 13, wherein said enzyme layer comprises deionized water, dipotassium hydrogen phosphate, lactic dehydrogenase.

20. The method for forming a potentiometric biosensor to detect lactate in food according to claim 13, wherein said oxide layer is formed by deposition of tin oxide on said substrate through magnetron sputtering at radio frequency (RF) power 50 W for 40 minutes.
